(19) United States
(12) Patent Application Publication
(10) Pub. No.: US 2006/0079719 A1
(43) Pub. Date: Apr. 13, 2006

(54) Aminated compounds bearing at least an allyl and a difluoromethyl and method used for synthesis thereof

(76) Inventors: Daniele Bonnet-Delpon, Paris (FR); Jean-Pierre Begue, Paris (FR); Julien Legros, Saint-Maur (FR); Benoit Crousse, Verriere-Le-Buisson (FR); Franck Meyer, Rueil-Malmaison (FR)

Correspondence Address: Jean Louis Seugnet;Rhodia Inc CN 7500, Intellectual Property Department, 259 Prospect Plains Road, Cranbury, NJ, 08512-7500, US

(21) Appl. No.: 10/511,487

(22) PCT Filed: Apr. 15, 2003
(86) PCT No.: PCT/FR03/01198

§ 371 Date: Oct. 15, 2004

(30) Foreign Application Priority Data
Apr. 16, 2002 (FR) 02 04725
Nov. 12, 2002 (FR) 02 14581

(51) Int. Cl. C07D 213/08; C07C 211/21

(52) U.S. Cl. 564/509; 546/250; 548/400

(57) ABSTRACT

The invention relates to aminated compounds bearing at least an allyl and a difluoromethyl and a method used for synthesis thereof. Said compounds comprise at least one carbon bearing: an amine function, an allyl or propargyl radical, a difluoromethylene group, and a hydrogen or a hydrocarbon radical, advantageously selected among those which are electron donors or hardly electroattractive (; 0. 1). The invention is useful for synthesis of fluorinated compounds.

The present invention relates to aminated compounds bearing at least one allyl and a difluoromethyl, and also to a method used for the synthesis thereof. The present invention relates more particularly to compounds capable of cyclizing by metathesis and to an original technique of allylation in the α-position of a difluoromethyl group or of an amine.

In recent times, novel fluorinated compounds have emerged in the pharmacy and agricultural fields. In particular, derivatives which are both difluoro-methylated and comprise a nitrogenous heterocycle are proving to be increasingly advantageous. Most commonly, the difluoromethylene groups are borne by the ring, while at the same time being exocyclic.

At this stage of the description, it is advisable to define a term which will be used subsequently in the description; this is the term “metathesis”. This term is known in linguistics, and from the very earliest of times, to denote a change in place of a letter, or of a syllable within a word or a group of words, in general within a word; this metathesis is referred to as “reverse metathesis” when there is an exchange between a letter, or a syllable of a word, with another letter, or another syllable of the same word. In French, one of the examples most commonly cited as metathesis is “réglisse” [liquorice] which comes from the Greek glucurrhiza (sweet root), with the phoneme “gl” changing place with the phoneme “rh”.

In the remainder of the text, the term “metathesis” will denote a phenomenon that is relatively far removed from the linguistic notion; this term will denote the replacement of two unsaturations with another unsaturation, with the ejection of an unsaturated molecule corresponding to the ejected portion. The two unsaturations may belong to one or two molecules.

In the field which is the subject of the present invention, some intramolecular metatheses have been demonstrated in the particular case where amino acids, or rather their esters bearing two unsaturations, have been subjected to Grubbs salts. The acid functions to which reference is made are oxygenated acid functions such as carboxylic functions, phosphoric functions or phosphonic functions, which functions have a strong electron-withdrawing capacity, in particular via a mesomeric effect.

Reference may be made, in particular regarding these cyclizing metatheses, to the articles by B. Mohr, D. M. Lynn, F. H. Grubbs, Organometallics 1996, 15, 4317; to P. Schwab, R. H. Grubbs, J. W. Ziller, J. Am. Chem. So. 1996, 118, 100. Reference may also be made to A. Furstner, M. Picquet, C. Bruneau, P. H. Dix-neuf, Chem. Commun. 1998, 1315.

One of the aims of the present invention is to provide compounds comprising two unsaturations and an amine function comprising in the β-position a difluoromethyl group and not comprising the acid functions (optionally in ester form) specified above, and in particular not comprising phosphonate functions or carboxylate functions in the β-position of the amine function.

Another aim of the present invention is to provide compounds of the type above, which are capable of being cyclized by intramolecular metathesis.

Compounds of this type are very difficult to obtain. In fact, the difluoromethylene group generally has a tendency to modify the reactivity of the functions to which it is close, which makes the problem more difficult to solve, all the more so since its influence is somewhat erratic.

For this reason, one of the aims of the present invention is to provide compounds comprising at least one carbon bearing:
an amine function;
a difluoromethylene group;
   and not bearing an esterified or nonesterified oxygenated acid function.

Another aim of the present invention is to provide a compound of the above type, characterized in that it is capable of cyclizing under the action of Grubbs complexes.

Another aim of the present invention is to provide a method capable of obtaining a compound of the above type, using a step consisting in grafting an allyl group onto a carbon bearing trivalent nitrogen and a difluoromethylene group.

Another aim of the present invention is a method which makes it possible to graft an allylated group onto an imine bearing on its carbon a difluoromethylated (aldimine or ketimine) group, the imine not bearing a phosphonated or carboxylated function, and similarly not comprising an electron-withdrawing oxygenated acid function.

These aims, and others which will subsequently emerge, are achieved by means of compounds comprising at least one carbon bearing:
an amine function;
an allyl or propargyl radical;
a difluoromethylene group; and
a radical advantageously chosen from those which are electron-donating or weakly electron-withdrawing (σₚ≦0.2, advantageously to 0.1) radicals, preferably alkyl (lato sensus), more preferably hydrogen; characterized in that the amine function bears a hydrocarbon-based radical advantageously bearing an unsaturation that is ethylenic in nature.

The latter radical is advantageously such that said ethylenic unsaturation is in the allylic, or homoallylic, position relative to the amine.

Advantageously, according to the present invention, the compound above has the following formula:

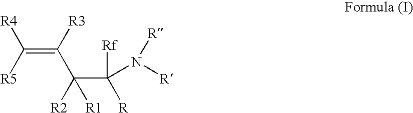

In this formula:
Rf represents a carbon radical bearing a difluoromethylene group providing the link with the rest of the molecule, advantageously of at most 15, preferably of at most 10, carbon atoms;
R₁ represents a hydrogen, an alkyl, including aralkyl, radical, preferably of 1 or 2 carbon atoms, or one of the specific radicals subsequently specified;
R₂ represents a hydrogen, an alkyl, including aralkyl, radical, preferably of 1 or 2 carbon atoms, or an aryl radical;
R₃ represents a hydrogen or a hydrocarbon-based radical, such as alkyl, including aralkyl, preferably of 1 or 2 carbon atoms, or an aryl radical, or forms, with R₄, an additional double bond so as to convert the allyl radical into a propargyl radical;
R₄ represents a hydrogen or a hydrocarbon-based radical such as aryl or alkyl, including aralkyl, preferably of 1 or 2 carbon atoms, or, with R₃, forms an additional double bond so that the ethylenic bond becomes acetylenic, making it possible to go from an allyl radical to a propargyl radical;
R₅ represents a hydrogen or a hydrocarbon-based radical such as an aryl radical or an alkyl, including aralkyl, radical, preferably of 1 or 2 carbon atoms;
R₅ may be an “Ar” group as described in the patent published under the number EP 565 607 B, patent titled protective group;
R₅ and R₄ may be fractions of said “Ar” group above, such that R₅ and R₄, and also the carbon which bears them, form a radical Ar as defined in the European patent above;
it being possible for one of R₁, R₂, R₄, R₃ and R₅ to be, in addition, chosen from trivalent, nitrile or acid functions, optionally and preferably in esterified form.

The carbon bearing the R_{f} group and the amine may be chiral and the compound above may be one of the chiral isomers.

The term “alkyl”, including aralkyl, is taken in its etymological sense of an alcohol from which the OH function has been removed; thus, an alkyl is a carbon, in general hydrocarbon-based, radical in which the function which has remained free is borne by an sp³ hybridization carbon, said sp³ carbon being itself linked only to hydrogen or carbon atoms.

It is preferable for at least 2, advantageously 3, preferably 4, of the radicals R₁ to R₅ to contain at most 5, advantageously at most 3, carbons; however, at least one of the radicals R₁ to R₅ may be such that the allyl radical corresponds to that of a heavy alcohol, for example of the aromatic series, of the terpenic series or of the steroid series.

Thus, one radical and at most 3 radicals, R₁ to R₅ may be aryl radicals, or homo- or heterocyclic, condensed or noncondensed, mono- or polycyclic radicals.

It is preferable for R₁ and R₂ to also be as small in volume as possible, if it is desired for the synthesis to be easy. Thus, it is preferable for R₁ and R₂ to be such that one of the 2 at least is hydrogen and the other is methyl or hydrogen.

As will be seen subsequently in the method of synthesis, it is advantageous for the radical R₃ to be other than carboxylic radicals, in particular other than carboxylic ester radicals, in order to avoid inopportune cyclizations during the synthesis.

The allyl radical formed by R₁ to R₅ and by the carbon atoms which bear them is advantageously palindromic such that the result of a synthesis which is allylic in nature and which gives the same results as the substitution is SN in nature or SN′ in nature.

The radical R is an electron-donating or weakly electron-withdrawing radical or group; that may be defined by indicating that, advantageously, σₚ is at most equal to 0.2. In general, R is chosen from alkyls, hydrogen and aryls. R is advantageously hydrogen, R′ may be a hydrogen, a protective group, an aryl or an alkyl, including aralkyl. Among the protective groups, mention should be made of the known protective groups, in amino acid synthesis, for amine functions, in particular groups for which the open bond is borne by a carbon in the allylic, benzylic or propargylic position.

R″ is an allyl radical, a hydrogen or a metal cation, or a fraction of metal cations, when the metal is polyvalent, as is preferred.

For the use in cyclizing metathesis, it is preferable for just one of R′ and R″ to be a homoallyl or allyl radical.

It should be noted here that the reaction not only respects the chirality of the initially chiral carbon atoms, but also makes it possible, with excellent diastereoisomeric excesses, to convert a prochiral carbon into a chiral carbon. The prochiral carbon is here the carbon bearing the imine function. The chiral inducer is advantageously the radical R′, which is then chosen so that R′ is itself chiral.

Advantageously, the chiral carbon (or one of the chiral carbons) of R′ is close to the imine function (i.e. separated, via the most direct path, by at most 3 ring-members, advantageously at most two, including atom, from the nitrogen of said imine function), preferably directly linked to the nitrogen of the imine function. To obtain a better chiral induction, it is preferable to have an at least bidentate system, the first prong being the nitrogen of the imine function and the second prong being a metalloid atom chosen from those of column V (nitrogen column) and of column VI (chalcogen column), this metalloid atom being placed in the beta-, gamma- or delta-position relative to the nitrogen of the imine function, so as to be able to form a 5-, 6- or 7-centered ring with a metal cation such as, for example, zinc.

Thus, the chiral groups bearing both, firstly, an amine function and, secondly, an alcohol function, or a function derived from the alcohol function, in particular ester and ether, constitute excellent chiral inducers for the reaction. When this group bears an aromatic, in particular phenyl ring, said aromatic ring being advantageously placed such that the nitrogen is in the benzylic position, this group can be considered to be a compound of formula R′-NH₂, and therefore to be a precursor of the compounds of formula II, in particular by means of condensation with a carbonyl derivative:

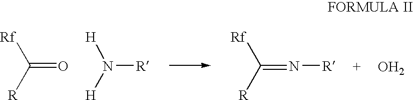

When the nitrogen of the imine of the compound of formula II is in the benzylic position, it can, after reaction, be readily freed of its protection R′ by techniques known in themselves, in particular by hydrogenolysis.

As will be seen in the examples, the diastereoisomeric excesses can reach a value substantially greater than 80%, and even 90%.

Thus, said chiral group is advantageously of formula:

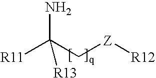

With R11 and R13 being different and chosen from alkyls, aryls and hydrogen, with q chosen from the integers 1 to 3 (i.e. 1, 2 or 3), with Z chosen from the metalloid elements of column V (nitrogen column) in the trivalent state advantageously bearing a hydrocarbon-based chain, and from the chalcogens. R12 is chosen from hydrogen and hydrocarbon-based, in particular aryl, alkyl and acyl, chains.

Advantageously, at least one of R11 and of R13 is chosen from aryls, advantageously homocyclic and/or containing six ring-members, preferably homocyclic and containing six ring-members.

Advantageously, q is equal to 1 or 2, preferably to 1.

The link between square brackets is an optionally mono-substituted, or even disubstituted, methylene link.

This methylene is preferably unsubstituted and therefore of formula —CH₂—.

R12 is advantageously alkyl or acyl.

As examples of molecules containing a chiral group, mention may be made of ephedrine and O-substituted derivatives of ephedrine, and glycinol derivatives.

It is desirable either for the chiral group to be part of a chiral polymer, or for it to be a molecule of at most 20 carbon atoms, advantageously at most 15 carbon atoms.

Thus, according to an advantageous embodiment of the present invention, R′ is a chiral radical of formula:

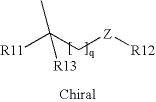

The number of carbons of the compounds of formula I according to the present invention is advantageously at most 30, preferably at most 20.

According to the present invention, insofar as it is directed toward the compounds according to the present invention, and only the compounds, it is preferable for at least one of R′ and of R″ to bear a function, or more exactly an unsaturation that is ethylenic in nature, in the allylic or homoallylic position, preferably allylic position.

The other of R′ and R″ is in general a protective group. It should be noted that the allyl radicals may be amine function-protecting groups. It should also be noted that the group R may be a specific alkyl, i.e. an allyl. In particular, according to the present invention, it has been possible, by reacting a derivative of formula II, where R is a halogen and where R′ and Rf have one of the values specified above, in particular when R′ is a protective radical, advantageously benzylic in nature, with 2 equivalents of allylic compounds such as allyl halides, to obtain a derivative comprising two allyl radicals, on the carbon bearing both the radical Rf and the amine function.

Advantageously, the Rf group, which advantageously comprises between 1 and 10, more preferably from 1 to 4, carbon atoms, corresponds to the formula below:
GEA-(CX₂)ₚ—
where:
the X, which may be similar or different, represent a chlorine, a fluorine or a radical of formula CₙF₂ₙ₊₁, with n an integer at most equal to 5, preferably to 2, with the condition that at least one of the X is fluorine, which fluorine is advantageously borne by the carbon bearing the open bond;
p represents an integer at most equal to 2;
GEA represents an electron-withdrawing group (i.e. sigma p greater than zero, advantageously than 0.1, preferably than 0.2) the possible functions of which are inert under the reaction conditions, advantageously fluorine or a perfluorinated residue of formula CₙF₂ₙ₊₁, with n an integer at most equal to 8, advantageously to 5;
   the total number of carbons of Rf being advantageously between 1 and 15, preferably between 1 and 10.

It is desirable for half, advantageously ¾ (rounded up to the nearest number if the fraction is not exact), preferably all, the X to be fluorines or “CₙF₂ₙ₊₁” radicals, preferably fluorines.

The Rf is most commonly perfluoroethyl, difluoromethyl, chlorodifluoromethyl and, especially, trifluoromethyl.

As was indicated above, another aim of the present invention is to provide methods comprising key steps for obtaining in particular compounds according to the present invention.

This aim is achieved by means of a process of allylation of a ketimine or of an aldimine linked directly to at least one perfluoromethylene (—CF₂)— ring-member, with the action of an allyl halide or pseudohalide, in the presence of an elemental metal, the oxidized form of which advantageously exhibits only one stable valency in the medium, which metal is at least as reductive as hydrogen, in other words, the redox potential (Mⁿ⁺+n e⁻ M⁰) is at most equal to 0 volt with respect to the hydrogen electrode, advantageously at most equal to that of zinc.

The ketimine or the aldimine advantageously corresponds to formula II below: 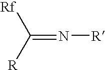
in which formula the radicals Rf, R and R′ are chosen from the values specified above, with in addition the possibility that R is chosen from the halogens.

The halide value for R is only of value when it is desired to obtain a diallyl derivative on the carbon bearing the future amine and the radical Rf.

According to the present invention, it is possible to use organometallic derivatives synthesized beforehand, and to bring the allylic organometallic derivative into contact with the imine only in a second step.

Thus, according to this procedure, the present invention is directed toward a method in which an organometallic is prepared by the action of an aryl halide on an element in the appropriate metal state. The appropriate metals for this operation are metals which have a redox potential that is sufficiently reductive to go from an allyl halide to an organometallic.

It is preferable to use metals which have only one stable valency state in the medium under consideration (besides the metal state of course). Mention may in particular be made of magnesium, zinc, nickel, cobalt, indium, or even lithium. However, it should be pointed out that alkali metals do not always give satisfactory results.

In fact, the reaction appears to be promoted when the amide corresponding to the reaction product is relatively undissociated, which explains why there is no competition between the first allylation and an N-allylation of the metal derivative derived from the first condensation.

The preferred metal is zinc.

However, according to the present invention, a different procedure is preferred, since it is liable to work more frequently. It involves bringing together the three reagents, namely the imine, the allyl halide and the metal, simultaneously: this is a technique referred to as the Barbier method.

According to this method, it is possible to mix the first two reagents and to only add the metal subsequently, but most commonly in fact, the reagents can be used simultaneously, the metal not reacting immediately with the other constituents of the reaction mixture. In fact, either the reaction is slow in starting, or it is desirable to add a zinc-activating element, such as traces of iodine or of silyl chloride, in order to activate the metal surface. An electrolytic activation can also be successfully carried out. Prior activation with dilute HCl is also possible.

The reaction mixture can advantageously be made conductive, for example by adding salts that are reputed to be clearly dissociated from the metal used during this allylation reaction. In particular, zinc bromide can be used.

The preferred solvents are polar aprotic solvents known in the synthesis of organometallics. More specifically, it is preferable for said polar aprotic solvent to have a significant dipolar moment. Thus, its relative dielectric constant E (epsilon) is advantageously at least equal to approximately 5, and advantageously at most equal to approximately 50. It is also preferable for the polar solvents used in the invention to be capable of clearly solvating the cations, which can be codified by the donor number D for these solvents, which is thus at least equal to 10, advantageously at least equal to 20. It is thus preferable for the donor number D for these solvents to be between 10 and 30, advantageously between 20 and 30.

Said donor number corresponds to the ΔH (variation in enthalpy), expressed in kilocalories, of the association of said polar aprotic solvent with antimony pentachloride. More specifically, reference may be made to the book by Christian Reichardt “solvents and solvent effects in organic chemistry—VCH p. 19, 1988”. Found therein is the definition of the donor number, which is defined as the negative (-ΔH) of the enthalpy (kilocalories per mole) of the interaction between the solvent and antimony pentachloride, in a dilute solution of dichloroethane.

The solvents that are particularly advantageous for the reaction targeted by the present invention are the solvents which are essentially amides, and more particularly formamides, including lactams and derivatives of urea (urea is here reputed to be a diamide of carbonic acid) and ethers, and in particular cyclic ethers.

The reaction works with all metal formulations, but the form of turnings works better than powders which itself works better than shot rather than in the form of powder.

This shot can, however, be used when the reaction can be carried out via the electrolyte route.

The leaving bearer allyl derivative which is condensed with the imine advantageously has formula III below:

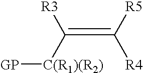

GP represents the leaving group, which is a halogen or pseudohalogen, advantageously halogen, such as chlorine, preferably bromine or iodine, more preferably bromine. The other radicals have already been defined above; they have the same values in formula III as in formulae I and II above.

A particular mention should be made when the group R₃ is a carboxylate, in particular alkyl carboxylate (ester). Specifically, in this case, the reaction should be carried out at low temperature so as to avoid a parasitic reaction, namely formation according to mecanistic pathways which have not been completely elucidated, namely the formation of a lactam which is none other than a pyrrolidone bearing a difluoromethylene group in the α-position of the nitrogen of the amide function.

Another point to consider is the choice of solvent. To avoid inopportune cyclization as specified above, it is also possible to choose a solvent which is a greater donor, and therefore which gives better salvation of the cations.

Thus, by choosing solvents with a donor number greater than that of THF (approximately 20), it is possible to carry out a reaction without parasitic cyclization. As seen in the table below, the amides are among the solvents which have the best, or at least the highest, donor number. These amides are particularly suitable for implementing the invention.

However, when the problem of a carboxylic group in the position of R₃ does not arise, then broader conditions can be used, in particular solvents having a lower donor number can readily be used.

TABLE

| Solvent | ε | DN | AN |
|---|---|---|---|
| DMSO | 48.9 | 29.8 | 19.3 |
| dimethyl sulfoxide | | | |
| CH₃CN | 38 | 14.1 | 18.3 |
| acetonitrile | | | |
| DMF | 36.7 | 26.6 | 16.0 |
| dimethylformamide | | | |
| NMP | 32.2 | 27.3 | 13.3 |
| dimethylpyrrolidone | | | |
| benzonitrile | 25.2 | 11.9 | 15.5 |
| DMPU | 36.1 | / | / |
| dimethylpropylene urea | | | |
| DMAC | 37.8 | 27.8 | 13.6 |
| dimethylacetamide | | | |
| anisole | 4.3 | / | / |
| xylene | 2.4 | / | / |
| diglyme | 5.7 | / | 9.9 |
| DMEU | 37.6 | / | / |
| dimethylethylene urea | | | |

The solvents targeted in the table are solvents which give good results, but it should be pointed out that DMSO is liable to give parasitic reactions in the presence of a reducing metal and that acetonitrile has a donor number that is a little low, which makes the reactions quite sluggish, and exhibits a risk of cyclization when R₃ is a carboxylic function.

The reaction is in general carried out at a temperature of between the melting point and the boiling point of the solvent, more generally between 0 and 50° C., when solvents having a donor number at least equal to 20 are used.

In the case of the amides, the reaction generally takes place at ambient temperature (i.e. at around 20° C. with a significant number).

When the reaction takes place on an allyl which is not palindromic, it may be mentioned, by way of indication, that, in general, the point of attachment of said allyl radical will be the one which corresponds to the most stable carbocation.

The latter point is not an indication regarding the mechanism, but simply a rule facilitating the provision of the reaction product.

The pressure is of only slight importance with regard to the reaction, unless it is desired to regulate the temperature by the boiling of a solvent, or if it is desired to eliminate one of the products of the reaction as it is formed.

The products obtained by means of the step of condensation of an imine with an allyl derivative can be pursued by means of other steps so as to give either a particularly advantageous component, or the diallyl derivatives which are subjects of the invention.

If cyclization, using the monoallyl products obtained in the preceding step, is desired so as to give a pyrrolidone, it is sufficient to carry out the reaction at a higher temperature or to take the monoallyl product and heat it for periods of time that are significantly longer than those required for the synthesis of said monoallyl product.

One of the simplest ways to obtain the diallyl products is to effect a release of the monoallyl amine formed and to react it, according to known techniques for amines, on the desired corresponding allyl derivative.

Thus, the step for allylation of the imine can be followed by a step for release of the amine, itself followed by a step for condensation of the amine on an allyl derivative comprising an appropriate leaving group, advantageously such as bromide and iodide.

The reaction regarding the first step can be symbolized by equation No. 1 below.

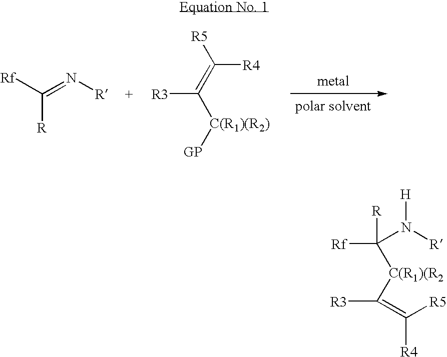

The allyl derivative to be condensed with the intermediate amine advantageously corresponds to formula IV.

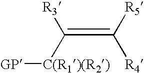

GP′ has the same values as GP, but the preference for halides is less marked than in the context of formula III.

R′₁ to R′₅ exhibit the same values and with the same preferred combinations as for the radicals ranging from R₁ to R₅.

The second allylation can be symbolized by equation 2 below: 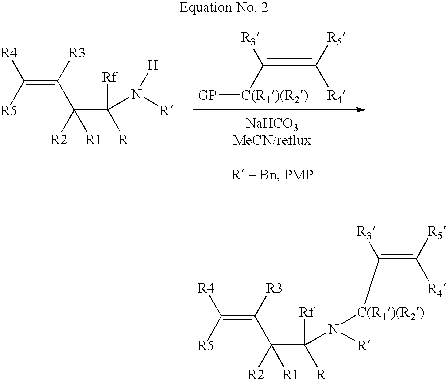
in which the product obtained during the step for condensation with the imine (either in the hydrogen form, or in an anionic form) is condensed with an allyl derivative comprising a leaving group. The operating conditions are gentle conditions, R′ is most commonly a protective group, which may advantageously be chiral and which also makes it possible to avoid a double allylation of the amine; this double allylation is in fact often promoted since the donor effect of the allyl group makes the amine nitrogen doublet more reactive.

The bases are the usual bases in the field. A bicarbonate has been represented in the equation, but other bases may be used, in particular non-quaternizable amines. In the example indicated, the solvent is acetonitrile, but other solvents may also be used.

The following nonlimiting examples illustrate the invention.

General Procedure

Unless otherwise specified, the procedure is according to that which follows. The imine and the allyl bromide used are dissolved in dimethylformamide, the allyl bromide being placed in an excess of approximately 30% relative to the imine. The solution is stirred at ambient temperature, and then zinc in the form of shavings is added in an excess of approximately 10% relative to the imine, and then a few drops of TMSCl are added to the mixture (the zinc may also be pre-activated with a dilute HCl solution, in which case the addition takes place at 0° C.). After 1 hour 30 minutes, the reaction mixture is cooled to 0° C. and then hydrolyzed with a saturated aqueous ammonium hydrochloride solution. The aqueous solution obtained is extracted with diethyl ether, three times successively. The organic phases are combined and washed with a saturated aqueous sodium chloride solution and then dried over magnesium sulfate. After evaporation of the solvent, the product is purified by silica gel chromatography by means of a mixture of ether of petroleum-ethyl acetate in a ratio of 80, 10 to 10. After evaporation-dilution of the eluent, the desired amine is obtained in the form of a colorless liquid.

Synthesis of Homoallyl and Homopropargyl Amines α-CF₃

Procedure

The ¹⁹F, ¹H and ¹³C NMR analyses were carried out on a Bruker 200 MHz device. The chemical shifts (δ) are given in ppm, according to CFCl₃ for the ¹⁹F NMR analyses, and according to TMS for the ¹H and ¹³C NMR analyses. All these analyses were carried out in CDCl₃ as solvent. The GPC analyses were carried out on an HP 4890 device equipped with an SE 30 apolar column (10 m). Besides the methallyl bromide which was distilled, all the reagents were used as they were, without any purification procedure.

Benzylimine Derivatives

Imine I corresponds to formula II, where Rf is a trifluoromethyl, where R is hydrogen and where R′ is benzyl:

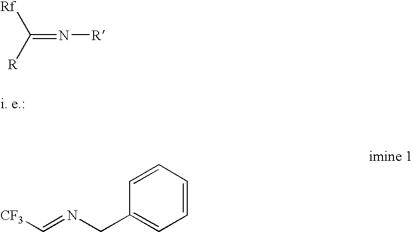

### EXAMPLE 1

### Synthesis of 4-benzylamino-5,5,5-trifluoropent-1-ene

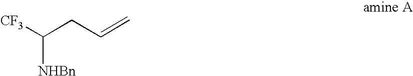

Imine 1 (1.03 g, 5.5 mmol) and the allyl bromide (865 mg, 7.15 mmol) are dissolved in DMF (10 ml) and placed under an argon atmosphere. The solution is cooled to 0° C. and stirred, and then the activated zinc (393 mg, 6.05 mmol) is added in a single step. The reaction mixture is kept at this temperature for 1 min and then left to return to ambient temperature (the progress of the reaction is monitored by GPC). After 1 hour 30 minutes, the reaction mixture is cooled to 0° C. and hydrolyzed with a saturated aqueous NH₄Cl solution (20 ml) and then extracted with diethyl ether (3×20 ml). The combined organic phases are washed with a saturated aqueous NaCl solution (50 ml), dried over MgSO₄ and filtered, and the solvents are then evaporated off. The crude product is purified by silica gel chromatography (petroleum ether/EtOAc, 90:10) so as to obtain the homoallyl amine in the form of a colorless liquid (1.16 g, 92%).

I.R. ν (cm⁻¹) 1644 (C═C);

¹⁹F NMR δ-74.9 (d, J_{H-F}=7.3 Hz, CF₃);

¹H NMR δ 1.5 (s, br, 1H), 2.3 (m, 1H), 2.5 (m, 1H), 3.1 (m, 1H), 3.8 (d, J=13 Hz, 1H), 4.0 (d, J=13 Hz, 1H), 5.1 (m, 1H), 5.2 (m, 1H), 5.7 (m, 1H), 7.2-7.4 (m, 5H);

¹³C NMR δ 33.3 (q, ³J_{C-F}, =2.2 Hz, CF₃CHCH₂), 52.1, 57.8 (q, ²J_{C-F}=28 Hz, CF₃CH), 118.8, 126.9 (q, ¹J_{C-F}=284 Hz, CF₃), 127.2, 128.2, 128.4, 133.1, 139.6.

Anal. calc. for C₁₂H₁₄F₃N (229.24): C, 62.87; H, 6.16; N, 6.11. Found: C, 62.65; H, 6.31; N, 5.99.

### EXAMPLE 2

### 4-benzylamino-2-methyl-5,5,5-trifluoropent-1-ene

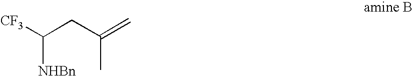

Using imine 1 (1.03 g, 5.5 mmol), the methallyl bromide (965 mg, 7.15 mmol) and Zn* (393 mg, 6.05 mmol), the product is obtained in the form of a pale yellow liquid (1.0 g, 75%).

I.R. ν (cm⁻¹) 1651 (C═C);

¹⁹F NMR δ-75.7 (d, J_{H-F}=7.0 Hz, CF₃);

¹H NMR δ 1.4 (s, 3H), 2.0 (dd, J=14 Hz, J=11 Hz, 1H), 2.3 (m, 1H), 3.0 (m, 1H), 3.6 (d, J=13 Hz, 1H), 3.9 (d, J=13 Hz, 1H), 4.6 (s, 1H), 4.7 (s, 1H), 7.1-7.3 (m, 5H);

¹³C NMR δ 21.2, 37.3 (q, ³J_{C-F}=2.7 Hz, CF₃CHCH₂), 52.6, 55.8 (q, ²J_{C-F}=28 Hz, CF₃CH), 114.8, 127.0 (q, ¹J_{C-F}=283 Hz, CF₃), 127.2, 128.3, 128.4, 139.4, 140.3.

### EXAMPLE 3

### 3-dimethyl-4-benzylamino-5,5,5-trifluoro-pent-1-ene

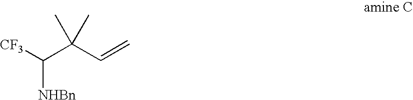

Using imine 1 (1.03 g, 5.5 mmol), the dimethylallyl bromide (1.07 g, 7.15 mmol) and Zn* (393 mg, 6.05 mmol), the product is obtained in the form of a pale yellow liquid (1.36 g, 96%).

I.R. ν (cm⁻¹) 1639 (C═C);

¹⁹F NMR δ-67.1 (d, J_{H-F}=8.4 Hz, CF₃);

¹H NMR δ 1.0-1.1 (m, 6H), 1.4 (s, br, 1H), 2.7 (q, J_{H-F}=8.3 Hz), 3.7 (d, J=13 Hz, 1H), 4.0 (d, J=13. Hz, 1H), 4.9 (dd, J=12 Hz, J=5 Hz, 2H), 5.8 (dd, J=16 Hz, J=9 Hz, 1H), 7.1-7.3 (m, 5H);

¹³C NMR δ 24.4, 40.1, 54.4, 66.3 (q, ²J_{C-F}=25 Hz, CF₃CH), 112.8, 127.2, 127.7 (q, ¹J_{C-F}=286 Hz, CF₃), 128.3, 128.4, 139.9, 144.3.

### EXAMPLE 4

### Ethyl 2-trifluoropropyl-(2-benzylamino)-propen-2-oate

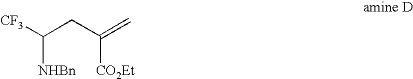

Using imine 1 (374 mg, 2 mmol), ethyl 2-(bromomethyl)-acrylate (502 mg, 2.6 mmol) and Zn* (143 mg, 2.2 mmol), the product is obtained in the form of a colorless liquid (1.0 g, 75%).

I.R. ν (cm⁻¹) 1632 (C═C), 1713 (CO₂Et);

¹⁹F NMR δ-75.3 (d, J_{H-F}=7.4 Hz, CF₃);

¹H NMR δ 1.2 (t, J=7.2 Hz, 3H), 2.4 (dd, J=15 Hz, J=10 Hz, 1H), 2.8 (dd, J=13 Hz, J=3.0 Hz, 1H), 3.3 (m, 1H), 3.7 (d, J=3.6 Hz, 1H), 4.0 (d, J=3.6 Hz, 1H), 4.2 (q, J=7.2 Hz, 2H), 5.6 (m, 1H), 6.3 (m, 1H), 7.2-7.4 (m, 5H);

¹³C NMR δ 3.9, 32.0 (q, ³J_{C-F}=2.7 Hz, CF₃CHCH₂), 52.0, 57.3 (q, ²J_{C-F}=27 Hz, CF₃CH), 60.7, 127.0 (q, ¹J_{C-F}=284 Hz, CF₃), 127.1, 128.1, 128.2, 136.2, 139.5, 166.3.

### EXAMPLE 5

### Synthesis of the Compound of Formula

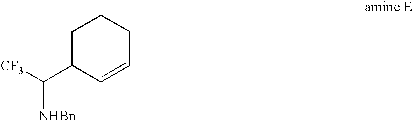

Using imine 1 (187 mg, 1 mmol), 3-bromocyclohexene (322 mg, 2 mmol) and Zn* (85 mg, 1.3 mmol), the product is obtained in the form of a colorless liquid (235 g, 87%).

I.R. ν (cm⁻¹) 1650 (C═C);

¹⁹F NMR 5-71.8 (d, J_{H-F}=8.7 Hz, CF₃);

¹H NMR δ 1.3-1.5 (m, 5H), 1.9 (m, 2H), 2.5 (m, 1H), 2.9 (qd, J=8.6 Hz, J=3.1 Hz, 1H), 3.7 (d, J=13 Hz, 1H), 3.9 (d, J=13 Hz, 1H), 5.6 (m, 1H), 5.8 (m, 1H), 7.1-7.3 (m, 5H);

¹³C NMR δ 21.8, 24.8, 27.2, 35.7 (q, ³J_{C-F}=1.6 Hz, CF₃CHCH), 53.0, 62.1 (q, ²J_{C-F}, =35 Hz, CF₃CH), 124.5, 125.3, 127.4 (q, ¹J_{C-F}=287 Hz, CF₃), 128.2, 128.3, 131.5, 139.8.

### EXAMPLE 6

### 4-benzylamino-5,5,5-trifluoropent-1-yne

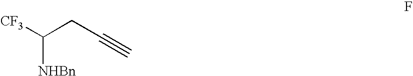

Using imine 1 (1.03 g, 5.5 mmol), 80% propargyl bromide in solution in toluene (1.06 g, 7.15 mmol) and Zn* (393 mg, 6.05 mmol), the product is obtained in the form of a colorless liquid (944 g, 76%).

I.R. ν (cm⁻¹) 3308 (C═CH);

¹⁹F NMR 5-75.1 (d, J_{H-F}=7.0 Hz, CF₃);

¹H NMR 52.0 (, br, 1H), 2.3 (t, J=2.6 Hz, 1H), 2.7 (ddd, J=17 Hz, J=7.6 Hz, J=2.6 Hz, 1H), 2.9 (ddd, J=17 Hz, J=4.8 Hz, J=2.8 Hz, 1H), 3.5 (m, 1H), 4.2 (d, J=13 Hz, 1H), 4.3 (d, J=13 Hz, 1H), 7.4-7.6 (m, 5H);

¹³C NMR δ 19.3 (q, ³J_{C-F}=3.2 Hz, CF₃CHCH₂), 51.9, 57.1 (q, ²J_{C-F}=28 Hz, CF₃CH), 71.2, 78.6, 126.0 (q, ¹J_{C-F}=284 Hz, CF₃), 127.3, 128.2, 128.4, 139.2.
Derivative of para-methoxyphenylimine

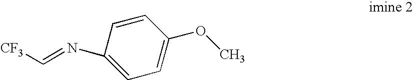

### EXAMPLE 7

### 4-(4-methoxyphenylamino)-2-methyl-5,5,5-trifluoropent-1-ene

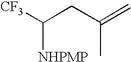

Using imine 2 (1.12 g, 5.5 mmol), the methallyl bromide (1.11 g, 8.25 mmol) and Zn* (393 mg, 6.05 mmol), the product is obtained in the form of a brown oil (1.28 g, 83%).

I.R. ν 1652 (C═C);

¹⁹F NMR δ-76.4 (d, J_{H-F}=6.6 Hz, CF₃);

¹H NMR δ 1.7 (s, 3H), 2.3 (dd, J=15 Hz, J=11 Hz, 1H), 2.6 (m, 1H), 3.2 (s, br, 1H), 3.7 (S, 3H), 3.9 (m, 1H), 4.8 (S, 1H), 4.9 (S, 1H), 6.6 (d, J=9.1 Hz, 2H), 6.8 (d, J=9.1 Hz, 2H);

¹³C NMR δ 21.5, 37.5 (q, ³J_{C-F}=1.9 Hz, CF₃CHCH₂), 55.3 (q, ²J_{C-F}=29 Hz, CF₃CH), 55.4, 114.6, 114.8, 114.9, 132.0 (q, ¹J_{C-F}=283 Hz, CF₃), 139.9, 141.1, 153.0.

Synthesis of the α,ω-Unsaturated Amines

General Procedure

The homoallyl or homopropargyl amine (1 mmol), the allyl bromide (3 eq, 3 mmol), NaHCO₃ (5 eq, 5 mmol) and KI (10 mol %, 0.1 mmol) are placed in acetonitrile (3 ml) and refluxed (the progress of the reaction is monitored by GPC). The reaction medium is allowed to return to ambient temperature, is hydrolyzed with brine (10 ml), and is then extracted with Et₂O (3×10 ml).

The combined organic phases are dried over MgSO₄ and filtered, and the solvents are then evaporated. The crude product is purified by silica gel filtration (petroleum ether) so as to obtain the α,ω-unsaturated amine.

### EXAMPLE 8

### Synthesis of the Compound of Formula

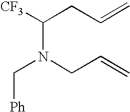

Amine A (229 mg, 1 mmol), the allyl bromide (363 mg, 3 mmol), NaHCO₃ (420 mg, 5 mmol) and KI (17 mg, 0.1 mmol) are heated for 3 days at the reflux of MeCN (3 ml). The product is obtained in the form of a colorless liquid (215 mg, 80%).

¹⁹F NMR 5-69.5 (d, J_{H-F}=8.7 Hz, CF₃);

¹H NMR δ 2.6 (m, 2H), 3.5 (m, 3H), 3.9 (d, J=14 Hz, 1H), 4.2 (d, J=14 Hz, 1H), 5.3 (m, 4H), 5.9 (m, 2H), 7.3-7.5 (m, 5H);

¹³C NMR δ30.9 (q, ³J_{C-F}=1.6 Hz, CF₃CHCH₂), 53.1, 53.8, 59.3 (q, ²J_{C-F}=25 Hz, CF₃CH), 117.2, 117.6, 127.0, 127.6 (q, ¹J_{C-F}=291 Hz, CF₃), 128.2, 128.7, 134.5, 136.3, 139.3.

### EXAMPLE 9

### Synthesis of the Compound of Formula

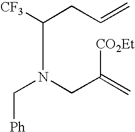

Amine A (229 mg, 1 mmol), ethyl 2-(bromomethyl)acrylate (290 mg, 1.5 mmol), NaHCO₃ (420 mg, 5 mmol) and KI (17 mg, 0.1 mmol) are heated for 3 days at the reflux of MeCN (3 ml). The product is obtained in the form of a colorless liquid (215 mg, 80%).

IR ν (cm⁻¹) 1716 (CO₂Et);
¹⁹_{φ}δ-69.2 (d, J=8.3 Hz, CF₃);

¹H NMR δ 1.2 (t, J=7.2 Hz, 3H), 2.3 (m, 2H), 3.1 (m, 1H), 3.4 (d, J=14 Hz, 1H), 3.6 (m, 2H), 3.9 (d, J=14 Hz, 1H), 4.1 (m, 1H), 4.2 (q, J=7.2 Hz, 2H), 4.96 (m, 1H), 5.7 (m, 1H), 5.8 (m, 1H), 6.1 (m, 1H), 7.1-7.3 (m, 5H); ¹³C NMR δ 14.0, 31.0 (q, ³J_{C-F}=1.5 Hz, CF₃CHCH₂), 50.9, 54.4, 59.8 (q, ²J_{C-F}=25 Hz, CF₃CH), 60.5, 117.3, 127.2, 127.3 (q, ¹J_{C-F}=290 Hz, CF₃), 128.2, 128.9, 134.4, 138.2, 138.4.

### EXAMPLE 10

### Synthesis of the Compound of Formula

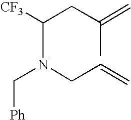

Amine B (243 mg, 1 mmol), the allyl bromide (363 mg, 3 mmol), NaHCO₃ (420 mg, 5 mmol) and KI (17 mg, 0.1 mmol) are heated for 3½ days at the reflux of MeCN (3 ml). The product is obtained in the form of a colorless liquid (226 mg, 80%).

¹⁹F NMR δ-69.1 (d, J_{H-F}=8.3 Hz, CF₃);

¹H NMR δ1.7 (s, 3H), 2.4 (dd, J=4.8 Hz, J=14.5 Hz, 1H), 2.6 (dd, J=10 Hz, J=14.5 Hz, 1H), 3.4 (m, 3H), 3.7 (d, J=14 Hz, 1H), 4.1 (d, J=14 Hz, 1H), 4.9 (d, J=14 Hz, 2H), 5.22 (m, 2H), 5.7 (m, 1H), 7.2-7.5 (m, 5H);

¹³C NMR δ21.5, 34.9, (q, ³J_{C-F}=1.6 Hz, CF₃CHCH₂), 53.0, 53.8, 57.3 (q, ²J_{C-F}=25 HZ, CF₃CH), 114.1, 117.7, 127.1, 127.8 (q, ¹J_{C-F}=291 Hz, CF₃), 128.2, 128.8, 136.5, 139.4, 141.0.

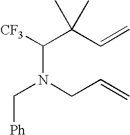

Amine C (257 mg, 1 mmol), the allyl bromide (363 mg, 3 mmol), NaHCO₃ (420 mg, 5 mmol) and KI (17 mg, 0.1 mmol) are heated for 5½ days at the reflux of MeCN (3 ml). The product is obtained in the form of a colorless liquid (124 mg, 42%).

¹⁹F NMR δ-59.5 (d, J_{H-F}=8.0 Hz, CF₃);

¹H NMR δ3.1 (m, 2H), 3.5 (q, J_{H-F}=8.0 Hz, 1H, CF₃CH), 3.7 (dq, J=14 Hz, J=1.5 Hz, 1H), 4.1 (m, 1H), 4.9 (m, 2H), 5.2 (m, 2H), 5.9 (m, 2H), 7.2-7.4 (m, 5H).

### EXAMPLE 11

### Synthesis of the Compound of Formula

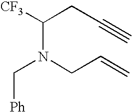

Amine F (227 mg, 1 mmol), the allyl bromide (363 mg, 3 mmol), NaHCO₃ (420 mg, 5 mmol) and KI (17 mg, 0.1 mmol) are heated for 4½ days at the reflux of MeCN (3 ml). The product is obtained in the form of a colorless liquid (211 mg, 79%).

IR ν (cm⁻¹) 3308 (C═C—H);

¹⁹F NMR δ-71.0 (d, J_{H-F}=7.9 Hz, CF₃);

¹H NMR β2.35 (ddd, J=17 Hz, J=5.4 Hz, J=2.9 Hz, 1H), 2.5 (ddd, J=17 Hz, J=9.0 Hz, J=2.6 Hz, 1H), 3.3 (m, 3H), 3.6 (d, J=14 Hz, 1H), 3.8 (d, J=14 Hz, 1H), 5.1 (m, 3H), 5.7 (m, 1H), 7.0-7.3 (m, 5H);

¹³C NMR δ 16.8 (q, ³J_{C-F}=2.2 Hz, CF₃CHCH₂), 53.4, 53.9, 58.6 (q, ²J_{C-F}=26 Hz, CF₃CH), 70.6, 80.0, 117.9, 126.5 (q, ¹J_{C-F}=289 Hz, CF₃), 127.1, 128.2, 128.6, 136.1, 139.0.

### EXAMPLE 12

### (2-methoxy-1-phenylethyl)(1-trifluoromethyl-but-3-enyl)amine

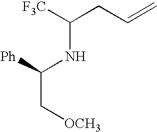

The chiral imine (423 mg, 1.83 mmol) and the allyl bromide (0.19 ml, 2.2 mmol) are dissolved in DMF (3 ml) and are placed under an argon atmosphere. The solution is stirred at ambient temperature and then zinc (155 mg, 2.38 mmol) in the form of shavings are added to the mixture, followed by a few drops of TMSCl. After 2 hours, the reaction mixture is cooled to 0° C. and then hydrolyzed with a saturated aqueous ammonium hydrochloride solution (20 ml). The aqueous solution obtained is extracted with diethyl ether (3×20 ml). The combined organic phases are washed with a saturated sodium chloride solution (50 ml) and then dried over magnesium sulfate and filtered. After evaporation of the solvent, the product is purified by silica gel chromatography by means of a mixture of petroleum ether-ethyl acetate (90:10) so as to obtain the homoallyl amine in the form of a colorless liquid (347 mg, 68%, e.d.=84%).

The reaction can be carried out in THF under the same conditions, but at reflux for 30 minutes. The yield is 85% (434 mg, e.d.=96%).

¹H NMR: (CDCl₃)

δ 7.36-7.12 (5H, m, H arom.), 5.75 (1H, m, CH═CH₂), 5.13 (2H, dd, J=9.6 Hz, J=16.8 Hz, CH═CH₂), 4.03 (1H, dd, J=5.6 Hz, J=7.6 Hz, CH—N), 3.30 (2H, m, CH₂OCH₃), 3.27 (3H, s, OCH₃), 2.95 (1H, m, CHCF₃), 2.39 (2H, m, CH₂—CH), 2.06 (1H, bl, NH).

¹³C NMR: (CDCl₃)

δ 139.6 (C arom.), 132.6 (CH═CH₂), 128.5, 127.9, 127.8 (C arom.), 126.2 (q, J=282 Hz, CF₃), 119.1 (CH═CH₂), 77.8 (CH₂—OMe), 60.2 (CH₃O), 58.7 (CHPh), 56.1 (q, J=28 Hz, CHCF₃), 33 (q, J=2 Hz, CH₂CH).

¹⁹F NMR: (CDCl₃)

δ-72 (d, J=8.1 Hz) 2%, −74.9 (d, J=8.5 Hz) 98%.

[α_{D}]=−86.4 (1.4/CHCl₃).

1-14. (canceled)

15. A compound comprising at least one carbon bearing:
an amine function;
an allyl or propargyl radical;
a difluoromethylene group; and
a hydrogen or a hydrocarbon-based radical which is electron-donating
or weakly electron-withdrawing with a σₚ≦0.2

16. A compound according to claim 15, wherein σₚ≦0.1.

17. A compound as claimed in claim 15, wherein the number of carbons of said compound is at most 30 carbon atoms.

18. A method for synthesizing a compound of formula I: 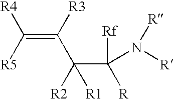
wherein:
Rf represents a carbon radical bearing a difluoromethylene group providing the link with the rest of the molecule, of at most 15 carbon atoms;
R₁ represents a hydrogen, an alkyl or aralkyl radical;
R₂ represents a hydrogen, an alkyl, aralkyl, or an aryl radical;
R₃ represents a hydrogen, alkyl, including aralkyl, an aryl radical, or forms, with R₄, an additional double bond so as to convert the allyl radical into a propargyl radical;
R₄ represents a hydrogen, aryl, alkyl, aralkyl, or, with R₃, forms an additional double bond so that the ethylenic bond becomes acetylenic, making it possible to go from an allyl radical to a propargyl radical;
R₅ represents a hydrogen, an aryl, an alkyl, or aralkyl radical;
R₅ and R₄ optionally being fractions of said “Ar” group above, such that R₅ and R₄, and also the carbon which bears them, form a radical Ar;
optionally, one of R₁, R₂, R₄, R₃ and R₅ being a specific trivalent, nitrile or acid function, optionally in esterified form;
R′ is a hydrogen, a protective group, an aryl, an alkyl, or aralkyl, optionally, a chiral alkyl or aralkyl; and
R″ is an allyl radical, a hydrogen, a metal cation, or a fraction of metal cations when the metal is polyvalent;
said method comprising the step of:
a) reacting an allyl organometallic on an imine bearing difluoromethylene groups, and
b) recovering the product obtained in step a).

19. The method as claimed in claim 18, wherein said organometallic is prepared in situ, according to a “Barbier” technique.

20. The method as claimed in claim 18, wherein the imine is of formula (II): 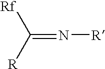
wherein R is halogen, a hydrocarbon-based radical which is electron-donating or weakly electron-withdrawing radical, and Rf is as defined above and wherein R′ is an alkyl, aralkyl, chiral, or protective group.

21. The method as claimed in claim 18, wherein the allyl radical of said organometallic corresponds to formula III: 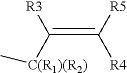
wherein R1, R2, R3, R4, and R5 are as defined above.

22. The method as claimed in claim 18, wherein the reaction of step a) is carried out in a polar aprotic solvent for which the donor number is at least equal to 10, optionally at least equal to 20.

23. The method as claimed in claim 18, further comprising the step of N-allylation, by means of the action of an allyl derivative of formula IV on the free amine.

24. The method as claimed in claim 18, wherein the Rf group corresponds to the formula below:
GEA-(CX₂)ₚ—
wherein:
the X, which are identical or different, represent a chlorine, a fluorine or a radical of formula CₙF₂n+₁, with n an integer at most equal to 5, with the proviso that at least one of the X is fluorine, which fluorine is optionally borne by the carbon bearing the open bond;
p represents an integer equals to 1 or 2; and
GEA represents an electron-withdrawing group with a sigma p greater than zero, the possible functions of which are inert under the reaction conditions; and
the total number of carbons of Rf being between 1 and 15.

25. The method as claimed in claim 24, wherein the total number of carbons of the radical Rf is between 1 and 4.

26. The method as claimed in claim 24, wherein the radical Rf corresponds to the formula: Cᵥ,Fᵥ₊₁, wherein v is an integer ranging from 1 to 10.

27. The method as claimed in claim 24, wherein the radical Rf is a difluoromethyl (CHF₂) radical or a trifluoromethyl radical.

28. A process for cyclizing metathesis, comprising the step of using as substrate a compounds of formula (I): 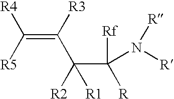
wherein:
wherein:
Rf represents a carbon radical bearing a difluoromethylene group providing the link with the rest of the molecule, of at most 15 carbon atoms;
R₁ represents a hydrogen, an alkyl or aralkyl radical;
R₂ represents a hydrogen, an alkyl, aralkyl, or an aryl radical;
R₃ represents a hydrogen, alkyl, including aralkyl, an aryl radical, or forms, with R₄, an additional double bond so as to convert the allyl radical into a propargyl radical;
R₄ represents a hydrogen, aryl, alkyl, aralkyl, or, with R₃, forms an additional double bond so that the ethylenic bond becomes acetylenic, making it possible to go from an allyl radical to a propargyl radical;
R₅ represents a hydrogen, an aryl, an alkyl, or aralkyl radical;
R₅ and R4 optionally being fractions of said “Ar” group above, such that R₅ and R4, and also the carbon which bears them, form a radical Ar;
optionally, one of R₁, R₂, R4, R₃ and R₅ being a specific trivalent, nitrile or acid function, optionally in esterified form;
R′ is a hydrogen, a protective group, an aryl, an alkyl, or aralkyl, optionally, a chiral alkyl or aralkyl; and
R″ is an allyl or homoallyl radical.

29. The process as claimed in claim 28, wherein just one of R′ and R″ is a homoallyl or allyl radical.
